(19) United States
(12) Patent Application Publication
(10) Pub. No.: US 2015/0101224 A1
(43) Pub. Date: Apr. 16, 2015

(54) Wrist Band

(71) Applicant: Brenmoor Limited, Yorkshire (GB)

(72) Inventors: Paul Brennan, Yorkshire (GB); Michael Moorhouse, Yorkshire (GB)

(73) Assignee: Brenmoor Limited, Yorkshire (GB)

(21) Appl. No.: 14/053,826

(22) Filed: Oct. 15, 2013

(51) Int. Cl. A61B 19/00; A44C 5/00; A44C 15/00; G09F 3/00

(52) U.S. Cl. CPC A61B 19/44; G09F 3/005; A44C 5/00; A44C 15/0085
USPC 40/633

(57) ABSTRACT

The invention provides for a medical wrist band, specifically for infants and babies, which is economical to manufacture, durable, easy on the skin, does not cause allergic reactions and cannot be removed and reapplied without being damaged.

### FIELD OF THE INVENTION

This invention relates to wrist bands, such as are nowadays applied to every patient upon admission to hospital.

### BACKGROUND OF THE INVENTION

Such wrist bands are well known and normally consist of a thermal, laser or inkjet printable plastic strap which is fastened to the patients' wrists detailing the person's ID and treatments.

The plastic strap is strong enough to survive several weeks of wear and tear on patients' wrists and normally is only replaced after a surgical intervention or other procedure that changes the patient's data.

It has been found, however, that the standard wrist bands are not suitable for infants and babies. The edges of the plastic can rub and cut the baby's skin leading to unnecessary medical complications.

A solution is known from US2006/0218837 A1 (J M Riley). It involves using a band of Velcro™ which can be looped around the infant's wrist and joined with a printable label attached to the outside of the Velcro band. The label has two slots by means of which it can be attached to the tongue which has the hook part of the Velcro on its inside. There is however, a problem with such Velcro strips. By definition, they are easily removable and the printable label can easily be removed from the Velcro band.

Since babies and infants are normally not in a position to offer explanations to staff, wrist band switches are not easily spotted and can lead to mistaken identities with all the consequences of such a mix up. What is more, the “Riley” solution is vulnerable to malicious interference which can go undetected for considerable time. A further factor speaking against the Velcro (Riley) solution is cost.

### SUMMARY OF THE INVENTION

The invention provides a wrist band as set out in this specification including the claims. It is directed at remedying the above mentioned problems. It is to create a wrist band which can be used for babies and infants, is economical to manufacture, durable, easy on the skin, does not cause allergic reactions and, most importantly, cannot be removed and reapplied without being damaged.

The band is made from latex free inert plastic foam sheeting section of about 1.5 mm thickness and may be any length or width required. The foam could also be substituted with another soft material such as fabric.

The complete wrist band consists of the main foam section of appropriate length, depending on the size of the patient, and a sticky plasticized printable label which can be preprinted or printed on demand. The sticky label is peeled off its backing strip and attached to the surface of one end of the shaped foam. The foam is wrapped around the patient's wrist and the sticky label wound over the top onto the foam. The label adheres to the foam and back onto its own surface which forms a continuous loop of the label around the patient's wrist to hold the whole together and ensuring only the foam is in contact with the wrist.

The glue on the plasticized strip is such that it will not peel away from the foam without tearing and showing signs of tampering. There is also the option to add security cuts into the strip which are designed to tear if the product is tampered with.

The invention has been described by means of the baby example. The extent of the invention covers adult patients as well as infants. It can be used anywhere where allergic reactions are likely that make the use of the standard wrist band unsuitable. Naturally, the band can also be used as ankle bands where appropriate, as with very small babies or patients with thinning and delicate skin due to age or illness.

### DESCRIPTION OF THE PREFERRED EMBODIMENTS

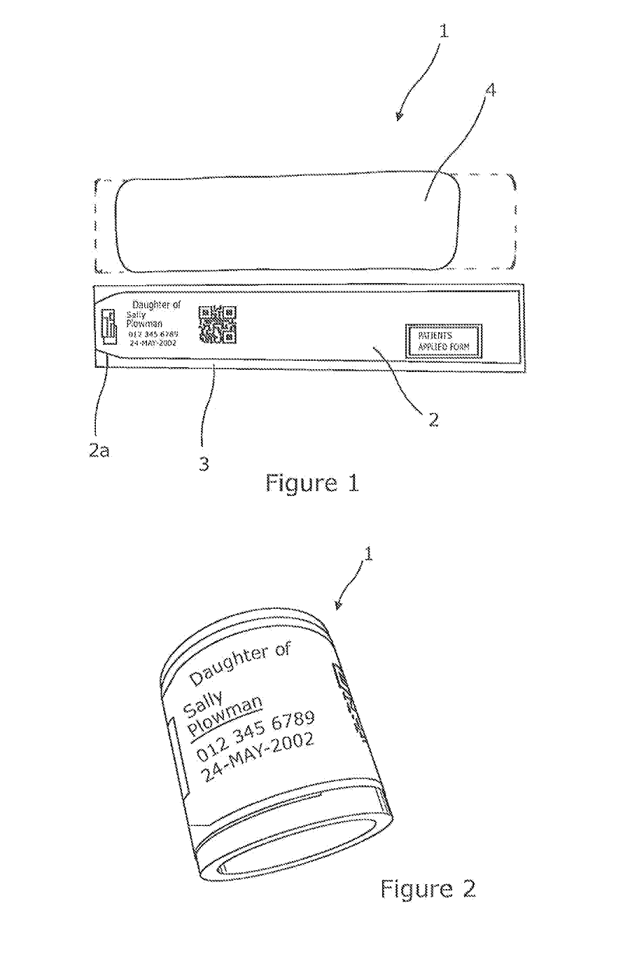

FIG. 1 shows the constituent parts of the baby-soft wrist band 1. The foam strip 4 is wrapped around the wrist or ankle of the infant, the label 2 is peeled off its backing strip 3 and wraps around the foam 4 and sticks onto itself. An easy way of applying the wrist band 1 is to partially peel the label 2 off its backing, press the peeled off end onto the foam 4 at one end and wrap the foam 4 around the body part by means of the end of the label 2 and stick down the label 2 onto itself thus wrapping the whole of the foam band 4 in the label 2. As can be seen from FIG. 1, the label 2 is longer than the foam band 4 to ensure that the label 2 can stick down upon itself and completely surrounding the foam band 4.

FIG. 2 shows the result of that operation, without the infant's body part being shown.

1. A hospital style identification limb band, comprising two strips of material to be joined together to form said limb band, wherein said first strip consists of an inert Latex free foam and said second strip consists of a plasticised strip in the form of an elongated label comprising an adhesive backing covered by a support layer and an area for printing information upon, wherein the foam strip is sufficiently wider than the plasticised strip to avoid said plasticised strip coming into contact with the patient's skin and wherein said limb band is assembled by partially peeling the plasticised strip off its backing support to expose part of the adhesive surface, pressing said inert foam onto the exposed adhesive surface, wrapping said foam around the wrist or ankle of a person to be identified and wrapping the plasticised strip around the foam and overlapping it onto the other end of said plasticised strip itself, thus forming said wrist band securely and irremovably on the wrist or ankle of the person to be identified and wherein said limb band cannot be reused after removal and any attempt at its removal will result in its destruction, since the adhesive is composed such that separation of the elongated plasticised strip from the foam causes tearing thereof.

2. (canceled)

3. A hospital style identification limb band according to claim 1, wherein the two strips of material are of equal length.

4. A hospital style identification limb band according to claim 1, wherein the two strips of material are of unequal length.

5. A hospital style identification limb band according to claim 1, wherein said elongated plasticised strip is printable on by laser, thermal or inkjet printers at or just before the moment of use.

6. A hospital style identification limb band according to claim 1, wherein said foam strip is fashioned from sheeting of about 2 to 3 mm thickness.

7. A hospital style identification limb band according to claim 1, wherein said foam is a closed cell blown low density polyethylene.

8. A hospital style identification limb band according to claim 1, wherein the band is adapted for use on infants and babies.

9. A hospital style identification limb band according to claim 1, wherein the overall length of the limb band is selectable so as to suit adults, teenagers and infants.

10. (canceled)

11. (canceled)

12. A hospital style identification limb band according to claim 1, wherein the foam section is substituted with one of: fabric, plastic and sponge.

13. A hospital style identification limb band according to claim 1, wherein the band is adapted for attachment to a wrist of the patient.

14. A hospital style identification limb band according to claim 1, wherein the band is adapted for attachment to an ankle of the patient.

15. A hospital style identification limb band according to claim 1, wherein the information printed upon the area for printing information is patient information.
